(19) United States
(12) Patent Application Publication
(10) Pub. No.: US 2026/0217775 A1
(43) Pub. Date: Jul. 30, 2026

(54) HYDROLYSATE OBTAINED FROM INSECTS AND METHOD FOR PRODUCING SAID HYDROLYSATE

(71) Applicant: Ynsect, Évry-Courcouroones Cedex (FR)

(72) Inventors: Lorena SANCHEZ, Juvisy-Sur-Orge (FR); Benjamin ARMENJON, Paris (FR); Constant MOTTE, Mons En Baroeul (FR); Bénédicte LORRETTE, Montlhéry (FR); Laura DARAI, Monthléry (FR)

(73) Assignee: Ynsect, Évry-Courcouronnes Cedex (FR)

(21) Appl. No.: 19/143,988

(22) PCT Filed: Dec. 28, 2023
(86) PCT No.: PCT/EP2023/087946

§ 371 Date: Jun. 27, 2025

(30) Foreign Application Priority Data
Dec. 30, 2022 (FR) FR2214712

(51) Int. Cl. C07K 14/435; A23K 10/14; A23K 10/20; A23K 50/80; C12P 21/06

(52) U.S. Cl. CPC C07K 14/43563; A23K 10/14; A23K 10/20; A23K 50/80; C12P 21/06

(57) ABSTRACT

The present invention relates to a hydrolysate obtained from insects, comprising at least 50% by weight of proteins, at least 7% by weight of lipids, at least 10.5% by weight of ash, the percentages by weight being indicated relative to the dry weight of the hydrolysate, and a solids content of between 15% and 35%, the percentage being given relative to the weight of the hydrolysate. The invention also relates to a method for preparing the hydrolysate, and to the use thereof in particular for improving the growth of fish.

### TECHNICAL FIELD OF THE INVENTION

The present invention relates to a hydrolysate obtained from insects and also to the method for preparing said hydrolysate and its use in animal feed and particularly fish feed.

Aquaculture plays a crucial role in global food security, because it is a sector that has seen steady growth for decades. Nevertheless, it is faced with several major challenges, particularly in terms of feed, with increasing intensification of production and the importance of using sustainable ingredients with a lower carbon footprint.

In fish feed manufacture, the need to reduce dependence on fish meal has led to the development of alternative ingredients, of marine or land origin. These new feeds, including functional supplements, can play a key role in the transition to more sustainable practices, thus reducing the environmental footprint and improving fish farmers' economic incomes.

Thus, there is a need to find alternatives which make it possible to optimize development and obtain optimal growth of fish.

The inventors' work made it possible to demonstrate that it was possible to develop a hydrolysate obtained from insects, making it possible to considerably improve fish growth performance, even when it is introduced in small quantities.

Furthermore, the inventors demonstrated that said hydrolysate also made it possible to improve fecal consistency. The latter parameter is important to consider in Recirculating Aquaculture Systems (RAS), because it has a substantial impact on water quality. Indeed, waste must be minimized and be capable of being disposed of easily by mechanical means to prevent degrading filters.

### DISCLOSURE OF THE INVENTION

The present invention relates to a hydrolysate obtained from insects, comprising at least 50% by weight of proteins, at least 7% by weight of lipids, at least 10.5% by weight of ash, the percentages by weight being given relative to the dry weight of the hydrolysate, and a dry matter content between 15 and 35% by weight, the percentage being given relative to the weight of the hydrolysate.

By “hydrolysate”, it is understood a composition which comprises hydrolyzed proteins. Hydrolyzed proteins are obtained by enzymatic protein hydrolysis. Enzymatic protein hydrolysis consists of cleavage of a peptide bond with an enzyme at a specific cleavage site triggering the release of small-sized proteins or amino acids.

The hydrolysate according to the invention is more particularly a hydrolyzed aqueous composition, i.e. it comprises a moisture content of 65 to 85%, preferably 70 to 80%, more preferably 75 to 80%. Throughout this application, “hydrolyzed aqueous composition” and “hydrolysate” are considered to be equivalent.

It should be noted that within the scope of this application, and unless otherwise stipulated, the value ranges indicated are understood to be inclusive of limits.

As indicated above, the hydrolyzed aqueous composition thus comprises 15 to 35% dry matter, preferably 20 to 32%, more preferably 20 to 25% by weight of dry matter relative to the weight of the hydrolysate.

It should be noted that within the scope of this application, the terms “percentage by weight” and “percentages by mass” are equivalent.

Preferably, the moisture content is determined according to the method from EC Regulation 152/2009, as detailed in Example 2.

Throughout the application, where no date is specified for a regulation, standard or directive, it consists of the regulation, standard or directive in force on the date of filing.

By “insects”, it is understood insects at any stage of development, such as an adult, larval or nymph stage. Preferably, the insects used in the method according to the invention are edible.

More particularly, the insects can be chosen from the group consisting of Coleoptera, Diptera, Lepidoptera, Isoptera, Orthoptera, Hymenoptera, Blattodea, Hemiptera, Heteroptera, Neuroptera, Ephemeroptera and Mecoptera, preferably, from Coleoptera, Diptera, Orthoptera, Neuroptera and Lepidoptera.

Preferably, the insects are chosen from the group consisting of Tenebrio molitor, Hermetia illucens, Galleria mellonella, Alphitobius diaperinus, Zophobas morio, Blattera fusca, Tribolium castaneum, Rhynchophorus ferrugineus, Musca domestica, Chrysomya megacephala, Locusta migratoria, Schistocerca gregaria, Acheta domesticus and Samia ricini.

More preferably, in the hydrolysate according to the invention, the insects are Coleoptera.

The Coleoptera preferably used belong to the Tenebrionidae, Melolonthidae, Dermestidae, Coccinellidae, Cerambycidae, Carabidae, Buprestidae, Cetoniidae, Dryophthoridae families, or mixtures thereof, even more preferably the insects belong to the Tenebrionidae family.

More preferably, they consist of the following Coleoptera: Tenebrio molitor, Alphitobius diaperinus, Zophobas morio, Tenebrio obscurus, Tribolium castaneum and Rhynchophorus ferrugineus, or mixtures thereof, even more preferably Tenebrio molitor and Alphitobius diaperinus.

The insects are preferably farmed and not taken from the natural environment.

For example, the insects are farmed in an insect farm. Farming insects in a specific farm makes it possible not only to control and eliminate the risks associated with insect-borne diseases, but also to limit the risks associated with insect-derived feed product toxicity due to the presence of insecticides, for example. Furthermore, farming makes it possible to control insect supply quality and to limit supply costs.

Advantageously, the hydrolysate according to the invention comprises a fiber content between 1 and 5% by weight relative to the dry weight of the hydrolysate.

Preferably, the fiber content is determined according to AOAC method 985.29, as detailed in Example 2.

Advantageously, the hydrolysate comprises between 2% and 5%, preferably between 2.5% and 4.5%, more preferably between 3% and 4% by weight of fiber relative to the dry weight of the hydrolysate.

Advantageously, the fiber comprises chitin.

Within the scope of this application, by “proteins”, reference is made to the quantity of crude proteins. Crude protein quantification is well known to a person skilled in the art. By way of example, mention may be made of the Dumas method or the Kjeldhal method. Preferably, the Kjeldhal method is used, as detailed in Example 2. Unless indicated otherwise, by “proteins”, reference is made not only to proteins but also to peptides and free amino acids.

Advantageously, the hydrolysate comprises between 50 and 80% by weight of proteins, preferably between 55 and 70%, more preferably between 60 and 65% by weight of proteins relative to the dry weight of the hydrolysate.

Advantageously, the hydrolysate comprises at least 20% of essential amino acids, preferably between 20% and 55%, more preferably between 40% and 50% by weight of essential amino acids, the percentages by weight being expressed relative to the total weight of proteins in the hydrolysate.

By “total weight of proteins” or “weight of proteins” without further indication on the nature of the proteins, it is understood the weight of crude proteins present in the hydrolysate. This therefore includes water-soluble and insoluble proteins.

Preferably, the essential amino acid content is determined according to the ISO 13903:2005 standard and EC Regulation 152/2009 (for tryptophan).

By “essential amino acid”, it is understood the following amino acids: histidine, isoleucine, leucine, lysine, methionine, phenylalanine, threonine, tryptophan and valine. It should be noted that, for some species, other amino acids may also be essential, for example arginine for 10 fish.

Advantageously, the hydrolysate comprises at least 1% by weight of free amino acids, preferably between 2 and 15%, more preferably between 3% and 11% of free amino acids, the percentages being given relative to the dry weight of the hydrolysate.

Preferably, the free amino acid content is determined according to the ISO 13903:2005 method, as detailed in Example 2.

The 5 most abundant total amino acids are advantageously glutamic acid, aspartic acid, leucine, lysine and tyrosine. These amino acids are of particularly interest for fish health (for example, intestinal health and hormone secretion for glutamic acid and muscle protein synthesis for leucine).

The hydrolysate according to the invention preferably comprises from 40 to 60% by weight of insoluble proteins, the percentage by weight being expressed relative to the total weight of proteins. Hence, the hydrolysate according to the invention comprises 40 to 60% by weight of water-soluble proteins, the percentage by weight being expressed relative to the total weight of proteins.

Advantageously, the hydrolysate according to the invention comprises water-soluble proteins, 75% of the water-soluble proteins having a size of less than 12, 400 g/mol, relative to the total weight of water-soluble proteins.

By “water-soluble proteins”, it should be understood, among proteins (or crude proteins), those that are soluble in a solution consisting of 30% acetonitrile, 70% ultrapure water and 0.1% trifluoroacetic acid (“ACN/water/TFA solution”), the percentages being percentages by volume relative to the total volume of solution, as detailed in Example 2.

By “insoluble proteins”, it should be understood proteins that are insoluble in the ACN/water/TFA solution, as detailed in Example 2.

Advantageously, the hydrolysate comprises a content greater than 80%, preferably between 85% and 98%, more preferably between 90% and 95% by weight of water-soluble proteins having a size of less than 12, 400 g/mol, relative to the total weight of water-soluble proteins.

Advantageously, the hydrolysate comprises a content greater than 40%, preferably between 50% and 70%, more preferably between 55% and 60% by weight of water-soluble proteins having a size of less than 555 g/mol, relative to the total weight of water-soluble proteins.

Preferably, the size of the proteins is determined by HPLC-SEC as detailed in Example 2.

The small size of the proteins of the hydrolysate according to the invention makes this hydrolysate highly digestible.

Advantageously, the hydrolysate has a pepsin and/or ileal digestibility greater than or equal to 95% by weight, preferably greater than or equal to 96% and more preferably greater than or equal to 98%, and more particularly greater than or equal to 99% by weight relative to the total weight of proteins.

Preferably, the pepsin digestibility is measured according to the methods AOAC 971.09, AOAC 992.15; AOAC 990.03 and AOCS Ba 4e-93, as detailed in Example 2. The ileal digestibility is measured according to the BOISEN and DUMAS methods, as detailed in Example 2.

As indicated above, the hydrolysate according to the invention comprises at least 7% by weight of lipids, expressed relative to the dry weight of the hydrolysate. Preferably, the hydrolysate comprises between 7% and 20% by weight of lipids, preferably between 8% and 18% by weight, more preferably between 9% and 16% by weight of lipids relative to the dry weight of the hydrolysate.

Methods for determining the fat (lipid) content are well known to a person skilled in the art. Preferably, this content will be determined by following the method of EC Regulation 152/2009, as detailed in Example 2.

As indicated above, the hydrolysate according to the invention comprises at least 10.5% by weight of ash, expressed relative to the dry weight of the hydrolysate. Advantageously, the hydrolysate comprises an ash content between 11% and 35% by weight, preferably between 12% and 30% by weight, more preferably between 15 and 28% by weight relative to the dry weight of the hydrolysate.

Preferably, the ash content is determined according to a method of EC Regulation 152/2009 as detailed in Example 2.

Advantageously, the hydrolysate according to the invention comprises one or more additives chosen from a preservative and/or a pH-reducing agent.

The aim of the additives added to the hydrolysate is to stabilize it. Hence, the hydrolysate can be stored for a period of more than 6 months, such as for example between 6 and 12 months at a temperature of 30 degrees Celsius.

Preferably, the preservative is chosen from sodium formate, sorbic acid, formic acid, potassium diformate, calcium formate, sodium bisulfate, potassium sorbate, acetic acid, sodium diacetate, calcium acetate and prenyl acetate.

The preservative is preferably introduced into the hydrolysate at a concentration of 0.05 to 3%, preferably from 0.1 to 2% by weight of the hydrolysate.

By “pH-reducing agent”, it should be understood a product capable of reducing the pH of an aqueous solution.

Preferably, the pH-reducing agent is chosen from an organic or inorganic acid, preferably, an inorganic acid, such as for example phosphoric acid, citric acid, fumaric acid, acetic acid, sorbic acid or propionic acid.

Advantageously, the pH-reducing agent is introduced at a necessary and sufficient quantity to lower the pH between 2 and 4, preferably between 2.5 and 3.5, more preferably between 2.8 and 3.2.

The present invention also relates to a method for preparing a hydrolysate from insects comprising the following steps:
a) Separating the cuticles and the soft part of the insects,
b) Separating the soft part of the insects into an aqueous fraction, an oily fraction and a solid protein fraction, and
c) Enzymatic hydrolysis of the solid protein fraction.

The method according to the invention makes it possible to obtain the hydrolysate according to the invention.

The hydrolysate and the insects are as described above, with their advantageous and preferred embodiments.

Thus, in the method according to the invention, enzymatic hydrolysis is carried out on the solid protein fraction and therefore after separating the soft part into three fractions. The inventors demonstrated that carrying out the hydrolysis step on the solid protein fraction, after separating the soft part into three fractions, had advantages compared to a method where the hydrolysis would be carried out before the step of separating into three fractions and particularly made it possible to improve the profitability of the method and to keep the other fractions unchanged.

Preferably, the method according to the invention further comprises a step of slaughtering the insects, prior to the step of separating the cuticles from the soft part.

This slaughtering step is described in more detail in step 1 of the method according to the invention detailed hereinafter.

The cuticle is the outer layer (or exoskeleton) secreted by the epidermis of insects. It is generally formed of three layers: the epicuticle, the exocuticle, and the endocuticle.

By “soft part”, it is understood insect flesh (particularly comprising the muscles and viscera) and fluids (particularly comprising biological fluids, water and hemolymph). In particular, the soft part does not consist of insect fluids.

The separation of the cuticles from the soft part of the insects can be carried out using any type of suitable separator, such as a belt separator or a twin-screw separator.

The separation of the cuticles from the soft part of the insects is described in more detail in step 2 of the method according to the invention detailed hereinafter.

Advantageously, the method according to the invention further comprises a step of maturing the soft part of the insects, between the step of separating the cuticles from the soft part and the step of separating the soft part of the insects into an oily fraction, a solid fraction and an aqueous fraction.

By “step of maturing the soft part of the insects”, it is more particularly understood a step during which the soft part of the insects undergoes heating and/or stirring.

This step is described in more detail in step 3 of the method according to the invention detailed hereinafter.

The aim of separating into three fractions is to recover three fractions from the soft part of the insects obtained in step 2 or optionally step 3, namely a solid fraction, an aqueous fraction, and an oily fraction.

This step of separating into three fractions is described in more detail in step 4 of the method according to the invention detailed hereinafter.

The solid protein fraction (also referred to as “protein cake”) is then subjected to an optional dilution step then to enzymatic hydrolysis.

The aim of the dilution step is to reduce the dry matter, preferably to a content between 15 and 35% by weight.

The enzymatic hydrolysis step is performed only on the solid fraction obtained from separating into 3 fractions. It preferably implements at least one proteolytic enzyme, preferably a protease, and more preferably at least two proteases, said proteases being of different types. In this application, the names or suffixes “peptidase” and “protease” are used indifferently to refer to an enzyme lyzing a peptide bond of proteins.

Advantageously, it implements an aminopeptidase and a serine endopeptidase.

This step of enzymatic hydrolysis of the solid protein fraction (protein cake) is described in more detail in step 6 of the method according to the invention detailed hereinafter.

The method according to the invention is described in more detail hereinafter.

### Detailed Method for Treating Insects According to the Invention

### Step 1: Slaughtering the Insects

This slaughtering 1 step can advantageously be performed by thermal shock, such as by boiling or by blanching. This step 1 makes it possible to slaughter the insects while lowering the bioburden (reduction of alteration and health risk) and inactivating the internal enzymes of the insects capable of triggering autolysis, and thus rapid browning thereof.

For boiling, the insects, preferably larvae, are thus boiled in water for 2 to 20 min, preferably, 5 to 15 min. Preferably, the water is at a temperature between 87 to 100° C., preferably 92 to 95° C.

The quantity of water introduced during boiling is determined as follows: the ratio of the volume of water in ml relative to the weight in g of insect is preferably between 0.3 and 10, more preferably between 0.5 and 5, even more preferably between 0.7 and 3, even more preferably in the order of 1.

For blanching, the insects, preferably larvae, are blanched in water or steam (steam nozzles or bed) at a temperature between 8° and 105° C., preferably between 87 and 105° C., more preferably between 95 and 100° C., even more preferably 98° C. or water at a temperature between 9° and 100° C., preferably between 92 and 95° C. (via spray nozzles) or in mixed mode (water+steam) at a temperature between 8° and 130° C., preferably between 9° and 120° C., more preferably between 95 and 105° C., even more preferably 98° C. When the insects are blanched only in steam, the blanching is advantageously carried out in forced-circulation steam blanchers (“forced steaming”). The residence time in the blanching chamber is between 5 seconds and 15 minutes, preferably between 1 and 7 min.

Advantageously, following the slaughtering step 1, the insects are directly used for the implementation of step 2 of separating the cuticles from the soft part of the insects, i.e. the insects are not subjected to any treatment, such as grinding, freezing or dehydration between step 1 and step 2.

Step 2: Separating the Cuticles from the Soft Part of the Insects

The aim of step 2 is to separate the cuticles from the soft part of the insects.

The separation of the cuticles from the soft part of the insects can be carried out using any type of adapted separator.

According to a first embodiment, the separation of the cuticles from the soft part is carried out using a filter press.

Advantageously, the filter press used in the method for treating insects according to the invention is a belt filter press.

A belt filter press has two perforated squeezing belts (also referred to as “filter cloths”). The insects are placed between the two perforated squeezing belts so that the soft part of the insects is pressed through the perforations of the squeezing belts, whereas the solid part of the insects remains between the 2 perforated squeezing belts.

A person skilled in the art is capable of determining the diameter of the perforations of the squeezing belts and also the pressure to be exerted, allowing the separation of the cuticles from the soft part of the insects.

According to a second embodiment, the separation of the cuticles from the soft part is carried out using a belt separator.

By way of example, a belt separator can comprise a squeezing belt and a perforated drum, the squeezing belt surrounding at least a portion of the perforated drum.

The squeezing belt allows the intake and application of insects against the perforated drum so that the soft part of the insects is pressed through the perforations of the drum, whereas the solid part of the insects (cuticles) remains outside the drum.

The cuticles can then be recovered using a scraping knife.

Regarding the pressure, a person skilled in the art is capable of determining the pressure to be exerted allowing the separation of the cuticles from the soft part of the insects.

According to a third embodiment, the separation of the cuticles from the soft part is carried out using a twin-screw separator. Such a separator comprises a twin-screw inside a perforated drum. The soft part of the insects passes through the perforations of the drum whereas the cuticles are conveyed along the two screws to outside the separator

Advantageously, the diameter of the perforations of the drums is between 0.5 and 3 mm, preferably between 1 and 2 mm.

This step of separating the insects differs from conventional pressing in that it allows a (clear) separation of the soft part and the cuticles of the insects and not a separation of a fluid from a solid fraction.

The soft part obtained in step 2 comprises between 20 and 50% by weight of lipids, preferably between 30 and 40% by weight of lipids relative to the dry weight of the soft part.

Furthermore, the soft part comprises at least 45%, preferably at least 48%, more preferably at least 50% by weight of proteins relative to the dry weight of the soft part.

### Step 3 (Optional): Maturing the Soft Part of the Insects

The soft part of the insects then optionally undergoes a maturing step in a tank.

Advantageously, the maturing is carried out for a duration between 15 minutes and 3 hours, preferably for 1 hour.

Advantageously, the maturing is carried out at a temperature between 65 and 100° C., preferably between 85 and 100° C., more preferably at a temperature of about 90° C.

This step may also be subjected to stirring.

This step makes it easier to separate the soft part of the insects in step 4 hereinafter.

Preferably, the method according to the invention comprises such a step.

In particular, no dilution of the soft part of the insects in a solvent such as water is necessary in this step.

Step 4: Separating the Soft Part into a Solid Fraction, an Aqueous Fraction and an Oily Fraction

The aim of this step is to recover three fractions from the soft part of the insects obtained in step 2 or 3, namely a solid fraction, an aqueous fraction, and an oily fraction.

According to a first embodiment, this step of separating the soft part is carried out in two sub-steps.

In the first sub-step, the soft part of the insects undergoes settling using a 2-phase settler, so as to obtain a solid fraction and a liquid fraction.

In the second sub-step, the liquid fraction undergoes centrifugation, so as to recover an oily fraction and an aqueous fraction.

Advantageously, in this second sub-step, a plate centrifuge is used.

According to a second embodiment of step 4, the soft part of the insects undergoes settling using a 3-phase settler, so as to directly obtain an aqueous fraction, an oily fraction and a solid fraction.

Adapted 3-phase settlers are, for example, Tricanter® from Flottweg, or 3-phase settlers from GEA, such as the CA 225-03-33 settler.

Advantageously, the separation of the soft part is carried out according to the second embodiment.

Indeed, using a 3-phase settler makes it possible to obtain particularly effective phase separation. More particularly, the solid fraction obtained has a high dry matter content, the aqueous fraction comprises a low content of insoluble sediments (from the solid fraction) and oil, and the oily fraction comprises a low content of insoluble sediments (from the solid fraction) and water.

### Step 5 (Optional): Dilution

The solid fraction obtained at the end of step 4 (protein cake) is then optionally diluted by adding water in order to reduce the dry matter content of said fraction. More particularly, the dilution step is carried out by adding water, such as tap water or freshwater. This step is preferably carried out under stirring. Furthermore, heating can also be applied, for example heating from 30 to 80° C., preferably from 40 to 70° C., more preferably from 50 to 70° C.

At the end of the dilution step, the diluted solid fraction comprises a dry matter content between 15 and 35% by weight, preferably between 20 and 25% by weight.

### Step 6: Enzymatic Hydrolysis

The aim of enzymatic hydrolysis is to reduce the size of the proteins in the protein cake to make them more soluble and digestible.

Enzymatic hydrolysis is performed with at least one proteolytic enzyme, preferably a protease. In this application, the names or suffixes “peptidase” and “protease” are used indifferently to refer to an enzyme lyzing a peptide bond of proteins.

Advantageously, hydrolysis is performed for a duration of 1 to 6 h, preferably for 2 to 4 h, at a temperature of 45 to 75° C., preferably 50 to 65° C. and at a pH between 6 and 8, preferably between 7 and 7.5.

Enzymatic hydrolysis can be performed with a single protease or alternatively with a mixture of enzymes containing at least one protease, more preferably a mixture of enzymes containing several proteases.

Preferably, the protease is chosen from the group consisting of aminopeptidases, metallocarboxypeptidases, serine endopeptidases, cysteine endopeptidases, aspartic endopeptidases, metalloendopeptidases.

Advantageously, the enzymes can be chosen from the following:

TABLE 1

| | | EC | | | |
|---|---|---|---|---|---|
| Enzyme (s) | Class | Number | Supplier | City | Country |
| Flavour- | Amino- | EC | Novozyme | Bagsvaerd | Denmark |
| zyme | peptidases | 3.4.11.1 | | | |
| Fungal | | EC | Bio-Cat | Troy | United |
| protease | | 3.4.11.1 | | | States |
| 500 | | | | | |
| ProteAX/ | | EC | Amano | Nagoya | Japan |
| AXH | | 3.4.11.1 | | | |
| Protex P | Serine | EC | Genencor | Leiden | Nether- |
| | endo- | 3.4.21 | Inter- | | lands |
| | peptidases | | national | | |
| | | | B.V. | | |
| Chymo- | | EC | Novozyme | Bagsvaerd | Denmark |
| trypsin | | 3.4.21.1 | | | |
| Protamex | | EC | Novozyme | Bagsvaerd | Denmark |
| | | 3.4.21 | | | |
| Elastase | | EC | Novozyme | Bagsvaerd | Denmark |
| | | 3.4.21.14 | | | |
| Trypsin | | EC | Novozyme | Bagsvaerd | Denmark |
| | | 3.4.21.36 | | | |
| Alcalase | | EC | Novozyme | Bagsvaerd | Denmark |
| | | 3.4.21.4 | | | |
| Papain | Cysteine | EC | Bio-Cat | Troy | United |
| | endo- | 3.4.22.2 | | | States |
| Bromelain | peptidases | EC | Bio-Cat | Troy | United |
| (ananase) | | 3.4.22.32 | | | States |
| Prolyve | Aspartic | EC | Lyven | Colombelles | France |
| NP | endo- | 3.4.23 | | | |
| Pepsin | peptidases | EC | Sigma | Saint- | France |
| | | 3.4.23.1 | Aldrich | Quentin- | |
| | | | | Fallavier | |
| Neutral | Metalloendo- | EC | Bio-Cat | Troy | United |
| protease | peptidase | 3.4.24.28 | | | States |
| Protex 50 | Endo- | EC | Genencor | Leiden | Nether- |
| FP | peptidase | 3.4.21 | Inter- | | lands |
| | | | national | | |
| | | | B.V. | | |
| Pan- | Exo- & | n.a.* | Lyven | Colombelles | France |
| crealyve | endo- | | | | |
| | peptidase | | | | |
| | (protease + | | | | |
| | amylase | | | | |
| | mixture) | | | | |
| Izyme BA | Aspartic | EC | Novozyme | Bagsvaerd | Denmark |
| | protease | 3.4.23 | | | |
| Sumizyme | Enzyme | n.a.* | Takabio- | Aichi | Japan |
| | mixture | | Shin | | |
| | | | Nihon | | |
| Neutrase | Zn-based | EC | Novozyme | Bagsvaerd | Denmark |
| | endo- | 3.4.24 | | | |
| | protease of β | | | | |
| | amylolique- | | | | |
| | faciens | | | | |
| Novozyme | Protease | n.a.* | Novozyme | Bagsvaerd | Denmark |
| 37071 | | | | | |

*n.a.: not applicable

Advantageously, the enzymatic hydrolysis step is carried out with a mixture comprising or consisting of an endopeptidase and aminopeptidase, more preferably a serine endopetidase and an aminopeptidase.

The enzyme or enzyme mixture is introduced at a quantity ranging from 2.5 to 31 g/kg relative to the dry weight of the solid protein fraction, preferably from 4 to 21 relative to the dry weight of the solid protein fraction.

In terms of enzymatic activity, the quantity of enzyme or enzyme mixture introduced corresponds to an activity between 40 and 5000 IU/kg relative to the dry weight of the solid protein fraction.

More particularly, the quantity of serine endopeptidase introduced corresponds to an activity between 40 and 60 IU/kg relative to the dry weight of the solid protein fraction.

Similarly, the quantity of aminopeptidase introduced corresponds to an activity between 2500 and 5000 IU/kg relative to the dry weight of the solid protein fraction.

Preferably, the method according to the invention further comprises a step of stabilizing the hydrolyzed solid protein fraction obtained in step c), by adding one or more additives chosen from a preservative and/or a pH-reducing agent.

The additives are as described above, with their advantageous and preferential embodiments.

The present invention also relates to the use of the hydrolysate according to the invention in an animal feed.

Preferably, the animals are chosen from domestic animals, grazing animals, poultry or aquatic animals.

Advantageously, the domestic animals are chosen from dogs, cats, birds, rodents, terrestrial reptiles, and fish.

Advantageously, the grazing animals are selected from cattle, sheep, goats, pigs, equids, camelids and cervids.

Advantageously, the poultry are chosen from chicken, turkey, duck, goose, pigeon, quail, pheasant and ostrich.

Advantageously, the aquatic animals are chosen from seabirds, cetaceans, marine reptiles, crustaceans (e.g. shrimp), fish, preferably farmed fish.

The farmed fish are preferably sea bass, sea bream, gilt-head bream, sturgeon, stone bass, panga, salmon, tilapia, turbot or trout.

Preferably, the farmed fish are chosen from Salmonids (Salmonidae) and particularly comprise salmon and trout.

Preferably, the fish belong to the Salmo, Salvelinus, Onchorynchus, and/or Hucho genus, more preferably Salmo.

The particularly preferred species according to the invention are: Salmo salar (Atlantic salmon), Salmo trutta (Brown trout or Sea trout), Oncorhynchus kisutch (Coho salmon), Oncorhynchus tshawytscha (Chinook salmon), Onchorynchus mykiss (Rainbow trout) and Salvelinus alpinus (Arctic char).

The farmed fish is a fish farmed in freshwater or saltwater ponds or cages. The environment, water quality and feed are controlled.

For example, “Recirculating Aquaculture System” closed-loop farming systems may be implemented in fish farming.

Preferably, in the use according to the invention, the feed is in the form of granules, flakes, paste, kibble, or treats.

Advantageously, the feed is in granule or flake form. This form is particularly adapted to feeding aquatic animals such as fish.

Such a feed comprises, in addition to the hydrolysate, other ingredients adapted to feeding fish. These ingredients can comprise fish oils, animal meal such as krill meal, fish meal, or squid meal; cereal meals such as wheat meal; oilseed meals such as soy meal, pea meal; vegetable oils such as rapeseed oil; gelling agents such as guar gum; protein concentrates; wheat or corn gluten; vitamin and mineral pre-mixes or any other ingredients adapted to feeding fish.

Feed in paste or kibble form is particularly adapted to feeding domestic animals such as dogs or cats.

Advantageously, in the use according to the invention, the feed comprises from 1% to 20% by weight of the hydrolysate, the percentage by weight being given relative to the weight of the feed.

Advantageously, the feed comprises between 1% and 10% by weight, of the hydrolysate preferably between 1% and 8% by weight, more preferably between 1% and 5% by weight of the hydrolysate, the percentage being given relative to the weight of the feed.

Fish feed is most often prepared in granule form.

The manufacture of granules is well known to a person skilled in the art. The granules can particularly be obtained by extrusion.

According to a first embodiment, the hydrolysate is mixed with the other ingredients making up the feed as illustrated above, before extrusion or shaping.

According to a second embodiment, in a first phase, the other ingredients of the feed are extruded then the hydrolysate is introduced in the form of a coating applied under vacuum.

Techniques for coating granules and the conditions to be implemented are well known to a person skilled in the art.

The use according to the invention is advantageously implemented to improve the growth of farmed fish.

Adding the hydrolysate according to the invention to the diet of fish makes it possible to significantly improve the body weight gain and/or size (length) of said fish, preferably the body weight gain and size of said fish as demonstrated in Example 3 and in FIG. 1.

More particularly, the use of the hydrolysate according to the invention makes it possible to:
increase weight gain,
increase the specific growth rate,
reduce the feed conversion rate, and/or
increase the slaughtering yield, of farmed fish, and particularly of salmonids.

Preferably, the use of the hydrolysate according to the invention makes it possible to increase weight gain, increase the specific growth rate, and reduce the feed conversion rate of farmed fish, and particularly of salmonids.

The hydrolysate according to the invention, the farmed fish and the salmonids are more particularly those described above, including their advantageous and preferred embodiment.

The use according to the invention is advantageously implemented for improving fecal consistency.

By “fecal consistency”, it should be understood the degree of cohesion of the materials making up feces. This consistency can be measured on a scale from 1 to 4 as indicated in Example 4.

By “improving consistency”, it should be understood increasing the cohesion of the materials making up feces, particularly increasing solid and firm fecal content (such as with a score of 1) and reducing liquid fecal content (diarrhea, runny digestate, scores of 3-4).

A high fecal consistency is of interest for an aquaculture application, which requires having a high water quality to avoid degrading biofilters.

The present invention also relates to an animal feed comprising 1 to 20% by weight of the hydrolysate according to the invention, the percentage by weight being given relative to the weight of the feed.

### BRIEF DESCRIPTION OF THE FIGURES

Other features and advantages of the invention will become apparent in the following examples, given by way of illustration, with reference to:

FIG. 1 which represents six diagrams (FIGS. 1a) to 1f)) comparing the overall growth performances of rainbow trout fed for 84 days with the control diet (CTRL) with those of rainbow trout fed with the experimental diet according to the invention (Hydrolysate). The diets and parameters measured are described in more detail in Example 3 hereinafter.

FIG. 1a) compares the final weight of the trout expressed in grams obtained according to the two diets tested;

FIG. 1b) compares the final length of the trout, expressed in centimeters, obtained according to the two diets tested;

FIG. 1c) compares the weight gain of the trout expressed as a percentage determined according to the two diets tested;

FIG. 1d) compares the specific growth rate of the trout expressed as a percentage per day determined according to the two diets tested;

FIG. 1e) compares the daily feed intake of the trout determined (as a percentage of the body weight per day, on the y-axis) according to the two diets tested;

FIG. 1f) compares the feed conversion rate of the trout determined according to the two diets tested.

FIG. 2, which is a bar diagram comparing the slaughtering yield in salmon when the latter is fed with the feed according to the invention comprising the hydrolysate obtained from insects, or with the control diet;

FIG. 3, which is a bar diagram comparing the fecal consistency in salmon when the latter is fed with the feed according to the invention comprising the hydrolysate obtained from insects, or with the control diet; with on the y-axis, the percentage fecal distribution according to consistency (i.e., score of 1 or 2).

FIG. 4 shows four diagrams (FIGS. 4a) to 4d)) comparing the overall growth performances of Atlantic salmon fed for 58 days with the control diet (CTRL2) with those of Atlantic salmon fed with the experimental diet according to the invention (Hydrolysate). The diets and parameters measured are described in more detail in Example 5 hereinafter.

FIG. 4a) compares the final weight of the salmon expressed in grams obtained according to the two diets tested;

FIG. 4b) compares the final length of the salmon, expressed in centimeters, obtained according to the two diets tested;

FIG. 4c) compares the specific growth rate of the salmon expressed as a percentage per day determined according to the two diets tested;

FIG. 4d) compares the feed conversion rate of the salmon determined according to the two diets tested.

### EXAMPLE 1: METHOD FOR PREPARING THE HYDROLYSATE ACCORDING TO THE INVENTION

Firstly, 1 kg of T. molitor larvae is oven-dried then decuticled through a twin-screw separator thus allowing separation of the cuticles and the pulp (flesh).

The pulp thus obtained is placed in a maturing tank for 1 hour at 90° C. under stirring. The heated pulp is then separated via a tricanter (3-phase settler) thus making it possible to obtain an oily fraction, an aqueous fraction and a solid protein fraction corresponding to a protein cake.

The protein cake is then diluted by adding water to reduce the dry matter from 42% to 22%.

The hydrolysis step is then carried out at 60° C. for 2 hours using the following enzymes:
Alkalase: introduced at a concentration between 40-60 IU/kg expressed relative to the dry weight of the protein cake;
Flavourzyme: introduced at a concentration between 2500-5000 IU/kg expressed relative to the dry weight of the protein cake.

The enzymes are then deactivated at 90° C. for 30 minutes and the hydrolysate thus obtained is cooled to 40° C. before being stabilized by adding additives making it possible to obtain a pH of less than 2.9. The additives used can comprise a preservative and/or a pH-reducing agent.

The preservative can be chosen from sodium formate, sorbic acid, formic acid, potassium diformate, calcium formate, sodium bisulfate, potassium sorbate, acetic acid, sodium diacetate, calcium acetate and prenyl acetate.

The pH-reducing agent can be chosen from an organic or inorganic acid, preferably an inorganic acid, such as for example phosphoric acid, citric acid, fumaric acid, acetic acid, sorbic acid, propionic acid.

Finally, a filtration step is carried out in order to remove residual cuticle particles.

The finished product obtained is a hydrolysate (or hydrolyzed aqueous composition) having a dry matter between 20-25%.

### EXAMPLE 2: CHARACTERIZATION OF THE HYDROLYSATE ACCORDING TO THE INVENTION

The hydrolysate obtained in Example 1 was characterized as follows.

### 1. Analyses.

### 1.1 Determination of Moisture Content

The moisture content was determined according to the method from EC Regulation 152/09.

### 1.2 Determination of Quantity of Proteins

The protein content was determined according to the Kjeldahl method, of EC Regulation 152/2009, with a protein conversion factor N of 6.25.

### 1.3 Determination of Quantity of Lipids

The quantity of lipids was determined according to EC Regulation 152/2009.

### 1.4 Determination of Quantity of Ash

The ash content was determined according to EC Regulation 152/2009.

### 1.5 Determination of Quantity of Fiber

The (soluble and insoluble) fiber content was determined according to AOAC method 985.29.

### 1.6 Determination of Quantity of Amino Acids

The quantity of total and free amino acids was determined according to the ISO 13903:2005 standard (for total and free amino acids except tryptophan) and EC Regulation 152/2009 (for tryptophan).

The quantity of free amino acids was determined according to the ISO 13903:2005 standard.

### 1.7 Determination of Pepsin Digestibility

Pepsin digestibility was determined according to the methods AOAC 971.09 (0.02% pepsin digestibility (Gravimetry)) and AOAC 992.15; AOAC 990.03; AOCS Ba 4e-93 (protein combustion).

### 1.8 Determination of Ileal Digestibility

Ileal digestibility was determined according to the BOISEN methods (for enzyme assay) and the Dumas method (for protein content).

### 1.9 Determination of Quantity of Soluble and Insoluble Proteins

The quantity of soluble proteins was determined by solubilizing said proteins in a solution consisting of 30% acetonitrile, 70% ultrapure water and 0.1% trifluoroacetic acid (“ACN/water/TFA solution”), these percentages being percentages by volume relative to the total volume of the solution, which forms the mobile phase before assay by HPLC-SEC (steric exclusion chromatography method known to a person skilled in the art).

The quantity of insoluble proteins was determined by the dry residue obtained after dissolution of a sample of the dried hydrolysate in ACN/water/TFA solution, relative to the initial dry weight.

### 1.10 Determination of Protein Size

The size of the proteins was determined by HPLC-SEC.

### 1.11 Determination of Quantity of Minerals

The content of each of the minerals listed in Table 8 below was determined using the ICP/AES (inductively coupled plasma atomic emission spectrometry) method.

### 1.12 Determination of Quantity of Vitamins

The thiamine hydrochloride (vitamin B1-HCl) content was determined according to the BS EN 14122-2014 method.

The vitamin B12 (cyanocobalamin) content was determined according to the AOAC 952.20 method.

The riboflavin (vitamin B2) content was determined according to the EN 14152:2014 method.

The niacin (vitamin B3) content was determined according to the EN 15652:2009 method.

The pantothenic acid (vitamin B5) content was determined according to AOAC method 2012.16.

The pyridoxine (vitamin B6) content was determined according to the EN 14164:2014 method.

The phylloquinone (vitamin K1) content was determined according to the EN 14148:2003 method.

### 2. Results

The characteristics of the hydrolysate according to the invention are shown in Tables 2 to 8 below.

TABLE 2
Hydrolysate composition

| | | Hydrolysate | | |
|---|---|---|---|---|
| | Unit | Minimum | Maximum | Mean* |
| Dry | % by weight | 23.9 | 31.1 | 24.6 |
| matter | relative to | | | |
| | weight of | | | |
| | hydrolysate | | | |
| Proteins | % by weight | 60.5 | 65 | 63.1 |
| | relative to dry | | | |
| Lipids | weight of | 9.9 | 15.5 | 13.0 |
| Ash | hydrolysate | 15.2 | 27.1 | 21.0 |
| Fiber | | 3.3 | 3.3 | 3.3 |

*mean results calculated on 20 hydrolysates, except for fiber where a single measurement was made.

### Proteins and Amino Acids

TABLE 3
Amino acid content in hydrolysate

| | | Hydrolysate | | |
|---|---|---|---|---|
| Total amino acids | Unit | Minimum | Maximum | Mean** |
| Cysteine + Cystine | g/100 g* | 0.66 | 0.69 | 0.68 |
| Methionine | g/100 g | 1.146 | 1.151 | 1.15 |
| Alanine | g/100 g | 3.09 | 3.54 | 3.32 |
| Arginine | g/100 g | 3.65 | 3.66 | 3.66 |
| Aspartic acid | g/100 g | 6.49 | 6.72 | 6.6 |
| Glutamic acid | g/100 g | 8.93 | 9.12 | 9.03 |
| Glycine | g/100 g | 2.88 | 3.01 | 2.95 |
| Histidine | g/100 g | 1.74 | 1.83 | 1.79 |
| Isoleucine | g/100 g | 3.08 | 3.22 | 3.15 |
| Leucine | g/100 g | 5.14 | 5.48 | 5.31 |
| Lysine | g/100 g | 4.35 | 4.60 | 4.48 |
| Phenylalanine | g/100 g | 2.85 | 2.96 | 2.91 |
| Proline | g/100 g | 3.76 | 3.87 | 3.81 |
| Serine | g/100 g | 2.91 | 2.92 | 2.92 |
| Threonine | g/100 g | 2.91 | 2.91 | 2.91 |
| Tyrosine | g/100 g | 4.13 | 4.47 | 4.3 |
| Valine | g/100 g | 3.68 | 3.92 | 3.8 |
| Total tryptophan | g/100 g | 1.06 | 1.13 | 1.09 |

*g/100 g: g/100 g of hydrolysate in dry weight
**mean results calculated on several hydrolysates

TABLE 4
Free amino acid content

| | | Hydrolysate | | |
|---|---|---|---|---|
| Free amino acids | Unit | Minimum | Maximum | Mean** |
| Cystine | g/100 g* | 0.07 | 0.17 | 0.12 |
| Methionine | g/100 g | 0.19 | 0.24 | 0.21 |
| Alanine | g/100 g | 0.44 | 0.56 | 0.50 |
| Arginine | g/100 g | 0.73 | 0.81 | 0.77 |
| Aspartic acid | g/100 g | 0.16 | 0.34 | 0.25 |
| Glutamic acid | g/100 g | 0.35 | 0.68 | 0.51 |
| Glycine | g/100 g | 0.12 | 0.16 | 0.14 |
| Histidine | g/100 g | 0.29 | 0.31 | 0.30 |
| Isoleucine | g/100 g | 0.43 | 0.62 | 0.53 |
| Leucine | g/100 g | 1.04 | 1.36 | 1.20 |
| Lysine | g/100 g | 0.52 | 0.54 | 0.53 |
| Phenylalanine | g/100 g | 0.48 | 0.58 | 0.53 |
| Proline | g/100 g | 1.12 | 1.23 | 1.17 |
| Serine | g/100 g | 0.33 | 0.40 | 0.36 |
| Threonine | g/100 g | 0.42 | 0.50 | 0.46 |
| Tyrosine | g/100 g | 0.53 | 0.77 | 0.65 |
| Valine | g/100 g | 0.62 | 0.87 | 0.74 |
| Total tryptophan | g/100 g | 0.19 | 0.23 | 0.21 |
| Ornithine | g/100 g | 0.05 | 0.06 | 0.05 |

*g/100 g: g/100 g of hydrolysate in dry weight
**mean results calculated on several hydrolysates

TABLE 5
Water-soluble protein size

| | | |
|---|---|---|
| % insoluble | % by mass expressed | 51.151 |
| proteins | relative to total | |
| | proteins | |
| % of proteins | % by mass expressed | 6.514 |
| having a size | relative to soluble | |
| >12.4 KDa | protein fraction | |
| % of proteins | % by mass expressed | 11.185 |
| having a size | relative to soluble | |
| between | protein fraction | |
| 12.4-6.5 KDa | | |
| % of proteins | % by mass expressed | 6.884 |
| having a size | relative to soluble | |
| between | protein fraction | |
| 6.5-1.4 KDa | | |
| % of proteins | % by mass expressed | 18.579 |
| having a size | relative to soluble | |
| between | protein fraction | |
| 1.4-0.555 | | |
| % of proteins | % by mass expressed | 56.839 |
| having a size | relative to soluble | |
| <0.555 KDa | protein fraction | |
| % of proteins | % by mass expressed | |
| having a size | relative to soluble | 93.487 |
| <12.4 KDa | protein fraction | |

TABLE 6
Digestibility

| Digestibility | Unit | Hydrolysate* |
|---|---|---|
| Pepsin | % | 98.1 |
| digestibility | | |
| Ileal digestibility | % | 97.2 |

*mean results calculated on several hydrolysates

### Vitamins and Minerals

TABLE 7
Vitamin content

| Vitamins | Unit | Hydrolysate* |
|---|---|---|
| Thiamine | mg/100 g | 0.3 |
| hydrochloride | | |
| (vitamin B1-HCI) | | |
| Vitamin B12 | μg/kg | 9.5 |
| (cyanocobalamin) | | |
| Riboflavin | mg/kg | 22.8 |
| (vitamin B2) | | |
| Niacin | mg/100 g | 19.0 |
| (vitamin B3) | | |
| Pantothenic Acid | mg/kg | 21.3 |
| (vitamin B5) | | |
| Pyridoxine | mg/kg | 4.4 |
| (vitamin B6) | | |
| Alpha-tocopheryl | mg/kg | 6.7 |
| acetate | | |
| (Vitamin E) | | |
| Phylloquinone | mg/kg | 0.1 |
| (vitamin K1) | | |

*mean results calculated on several hydrolysates

TABLE 8
Mineral content

| Minerals | Unit | Hydrolysate* |
|---|---|---|
| Zinc (Zn) | mg/kg | 218.8 |
| Iron (Fe) | mg/kg | 82.8 |
| Copper (Cu) | mg/kg | 35.9 |
| Calcium (Ca) | mg/kg | 899.6 |
| Magnesium (Mg) | mg/kg | 3958.2 |
| Manganese (Mn) | mg/kg | 20.9 |
| Phosphorus (P) | mg/kg | 52301.3 |
| Potassium (K) | mg/kg | 11046.0 |
| Sodium (Na) | mg/kg | 1129.7 |

*mean results calculated on several hydrolysate

### EXAMPLE 3: INTRODUCING THE HYDROLYSATE ACCORDING TO THE INVENTION INTO FEED FOR FISH (RAINBOW TROUT)

a. Materials and Methods

A fish meal-based diet (CTRL) was formulated with ingredients meeting the known nutritional needs of rainbow trout. Based on this diet, another diet comprising the hydrolysate obtained according to Example 1 was produced (see Table 9 below).

TABLE 9
Experimental diet composition

| | | Diet comprising |
|---|---|---|
| | | hydrolysate |
| Ingredients | | according to the |
| as %* | CTRL | invention |
| LT fish meal | 35.9 | 35.5 |
| Hydrolysate | 0 | 1.0 |
| Wheat gluten | 10.5 | 10.4 |
| Soy concentrate | 15.5 | 15.3 |
| Fish oil | 12.20 | 12.10 |
| Soy lecithin | 1.3 | 1.3 |
| Wheat meal | 16.6 | 16.4 |
| Premix¹ | 2.0 | 2.0 |
| Guar gum | 2.0 | 2.0 |
| Hemoglobin | 4.0 | 4.0 |
| Total | 100 | 100 |

*% of dry matter relative to total weight of composition
¹Premix: consists of a premix of vitamins and minerals comprising the following ingredients: Butylated hydrotoluene (BHT) (E 321) 20.000 mg, Colloidal silica (E 551b) 176.700 mg, Sepiolite (E 562) 370.600 mg, Vitamin B2 3.000 mg, Vitamin B12 10 mg, Nicotinamide (3a315) 20.000 mg, Folic acid (3a316) 1.500 mg, Vitamin D3 (3a671) 200.000 IU, Vitamin A (3a672a) 2,000,000 IU, Vitamin E (3a700) 10.000 mg, Vitamin K3 (3a710) 2.500 mg, Vitamin B1 (3a821) 3.000 mg, Vitamin B6/pyridoxine hydrochloride (3a831) 2.000 mg, D-calcium panthenate (3a841) 10.000 mg, Biotin (3a880 mg) 300 mg, Inositol (3a900) 50.000 mg, Betaine anhydrous (3a920) 50.000 mg, Iron (iron (II) sulfate, monohydrate) (3b103) 600 mg, Iodine (potassium iodide) (3b201) 50 mg, Manganese (manganese oxide (II) ) (3b502) 960 mg, Zinc (zinc sulfate, monohydrate) (3b605) 750 mg, Copper (copper sulfate, pentahydrate) (E 4) 900 mg, Selenium (sodium selenite) (E 8) 1 mg.

These two diets were manufactured by extrusion according to common techniques known to a person skilled in the art in order to obtain granules having a size of 2 to 4 mm.

During extrusion, the feed is dried and then cooled.

For granules according to the CTRL diet, the granules are obtained by extrusion of a composition prepared from LT fish meal, wheat gluten, soy concentrate, soy lecithin, wheat meal, premix, guar gum and hemoglobin in the quantities given in Table 8, which are mixed for extrusion. Then, following extrusion, after cooling the extruded granules, an additional 7% by weight of fish oil is added by vacuum coating.

For the granules according to the invention, the granules are obtained by extrusion of a composition prepared from LT fish flour, wheat gluten, soy concentrate, soy lecithin, wheat meal, premix, guar gum and hemoglobin in the quantities given in Table 8, which are mixed for extrusion. Then, following extrusion, after cooling the extruded granules, two successive coatings are carried out according to the following steps:
1^{st} step: adding the hydrolysate (4% by weight of the composition at a dry matter content of 25%, i.e. 1% of dry matter relative to the total weight of the granule composition) to the extruded granules by vacuum coating,
2^{nd} step: low-temperature drying of the granule coated with the hydrolysate,
3^{rd} step: adding an additional 7% by weight of fish oil by vacuum coating.
   b. Growth Performance Test

Each diet was tested in quadriplicate (4 tanks per diet).

The rainbow trout were acclimatized for 15 days before the start of the growth test, after which 25 trout with a mean weight of 20 to 30 g were distributed at random into each tank (20 tanks in total). The tanks used are cylindrical fiberglass (volume: 500 L) connected to a recirculating aquaculture system (RAS).

The tanks are supplied with freshwater at temperatures between 15±1° C., with dissolved oxygen levels greater than 7±1 mg/L. The ammonium and nitrite concentrations in the water were monitored daily to keep them below toxic values. The fish are subjected to a light/dark photoperiod cycle of 12 hours.

The fish were fed by hand to apparent satiety once a day for 84 days (between 8 am and 9 am) up to a maximum of 3% of the daily biomass feed intake.

After 84 days of experimental feeding, all fish from each reservoir were sampled, anesthetized with MS222 and growth performance was evaluated.

### Parameters Measured for Growth Evaluation:

IBW (g): Initial body weight
FBW (g): Final body weight

$$SGR ( \% / day ) : Specific growth rate = [ ( In FBW - In IBW ) / days ] \times 100$$

ITL (cm): Initial total length
FTL (cm): Final total length

$$DFI ( \% body weight / day ) : Daily feed intake = ( gross feed intake / ( IBW + FBW ) / 2 / days ) \times 100$$

FCR: Feed conversion rate=[total feed intake (g)/WG (g)]

$$WG ( \% ) : Weight gain = [ ( FBW - IBW ) / IBW ] \times 100$$

### Results:

TABLE 10
Results on fish growth performance

| Parameter | CTRL | Hydrolysate* |
|---|---|---|
| IBW (g) | 37.06 ± 0.19 | 37.18 ± 0.38 |
| ITL (cm) | 15.02 ± 0.04 | 14.99 ± 0.01 |
| FBW (g) | 326.41 ± 10.75 | 340.21 ± 1.91* |
| FTL (cm) | 28.21 ± 0.18 | 28.47 ± 0.06* |
| WG (%) | 780.74 ± 26.79 | 815.73 ± 35.43 |
| SGR (%/day) | 2.59 ± 0.04 | 2.65 ± 0.07 |
| DFI | 1.47 ± 0.05 | 1.48 ± 0.04 |
| FCR | 0.87 ± 0.04 | 0.84 ± 0.03 |

*By “Hydrolysate”, it is understood the diet comprising the hydrolysate according to the invention.

The values are expressed as a mean±standard deviation (n=4).

The asterisks indicate significant differences between the experimental groups and the CTRL control group (Student's t-test, P<0.05; n=4).

### Conclusion:

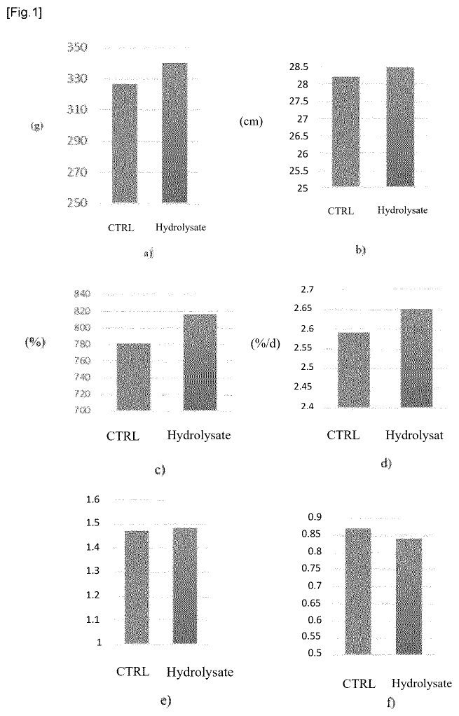

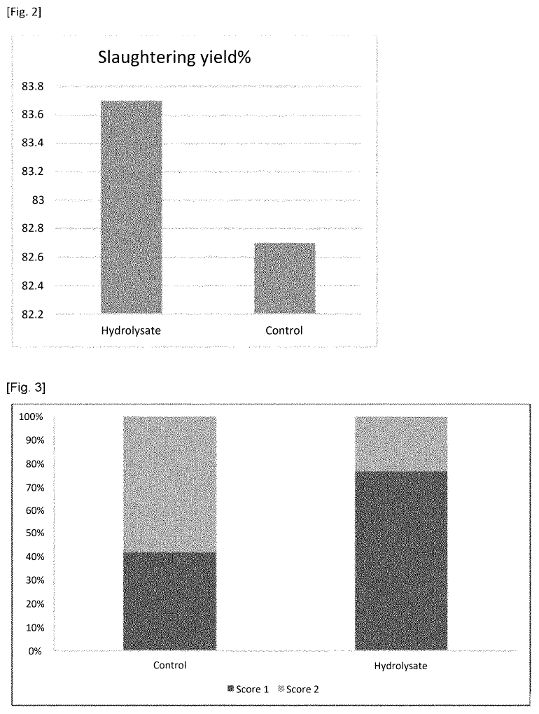

After 84 days of experimental feeding, it is observed that for an equivalent daily intake, the body weight and body length of the fish fed with the granules according to the invention increased significantly relative to the fish fed with CTRL granules (see FIG. 1).

Thus, the inventors demonstrated that use of the hydrolysate according to the invention at a very low dosage (1% dry matter) has a significant impact on fish growth.

### EXAMPLE 4: INTRODUCING THE HYDROLYSATE ACCORDING TO THE INVENTION INTO FEED FOR FISH (SALMON)

a. Materials and Methods

The hydrolysate described in Examples 1 and 2 is used. Commercially available 2 mm feed granules from Skretting AS (RCX) were used as a basis for producing two diets, detailed below. These granules particularly comprise 49% by weight of proteins, 22% by weight of lipids and 9% by weight of ash. Based on this formulation, a test diet was formulated, wherein 1% of hydrolysate was added by coating.

Finally, the two diets were prepared by coating the granules with water to stabilize the mean water content at about 13% by weight relative to the total weight of granules.

The tests were carried out on salmon. In total, 320 Atlantic salmon with a mean weight of 17 g were distributed at random into 6 tanks with a volume of 80 L (mean weight of 16.63-16.70 g per tank). Each diet was fed to three tanks in excess of appetite.

The experiment was conducted for 3 weeks.

### Growth Evaluation

All fish were weighed individually at the start of the experiment and at harvest (end of experiment).

### Fecal Evaluation

At the end of the experiment, 10 fish per tank were selected at random for fecal appearance.

Fecal consistency was visually analyzed and evaluated on a scale from 1 to 4, where the score of 1: solid and firm, the score of 2: semi-solid, soft digestate, the score of 3: diarrhea, runny digestate, the score of 4: yellow or non-white slurries of non-food materials/no digestate.

The digestate corresponds to the residual material derived from methanization (anaerobic digestion).

b. Results

### Growth Evaluation

Body weight increased 2.2-fold during the experimental period, from 17 g to 38.5-39.5 g, corresponding to a growth rate (SGR) of 3.48-3.58. As shown in FIG. 2, the slaughtering yield was significantly lower for the control (82.7%) relative to the diet comprising hydrolysate (83.7%).

### Fecal Evaluation

The softest consistency is observed for the CTRL group (Table 11, FIG. 3, left bar).

TABLE 11
Mean fecal score.

| | Hydrolysate* | Control |
|---|---|---|
| Fecal score | 1.3 ± 0.1^{b} | 1.6 ± 0.1ª |

*By “Hydrolysate”, it is understood the diet comprising the hydrolysate according to the invention.

As can be observed in FIG. 3 and Table 11, including a hydrolysate in the diet of salmon significantly improved fecal consistency relative to the control diet. Indeed, relative to the control diet, the diet comprising T. molitor hydrolysate improved the proportion of solid feces (score of 1), between 70% and 80% respectively, compared to 40% for the control diet.

The lower incidence of semi-solid stools conveys the interest of the hydrolysate according to the invention in animal feed supplementation, particularly for an application in aquaculture, which requires a high water quality to prevent biofilter degradation.

The experimental data generated in this example support the statement that animal feed supplementation with the hydrolysate according to the invention significantly improves fecal consistency.

### EXAMPLE 5: INTRODUCING THE HYDROLYSATE ACCORDING TO THE INVENTION INTO FEED FOR FISH (SALMON)

a. Materials and methods

The method described in Example 1 was reproduced and made it possible to obtain a hydrolysate having the following characteristics:

TABLE 12
Hydrolysate composition

| | Unit | Hydrolysate |
|---|---|---|
| Dry | % by weight | 25% |
| matter | relative to | |
| | weight of | |
| | hydrolysate | |
| Proteins | % by weight | 62.4% |
| Lipids | relative to dry | 10% |
| Ash | weight of | 21.6% |
| Fiber | hydrolysate | 3.2% |

A control diet (CTRL2) was formulated with ingredients meeting the known nutritional needs of salmon. Based on this diet, another diet comprising the hydrolysate described in Table 12 was produced (see Table 13 below).

TABLE 13
Experimental diet composition

| | | Diet comprising |
|---|---|---|
| | | hydrolysate |
| Ingredients | | according to the |
| as %* | CTRL2 | invention |
| Soy protein | 26.0 | 25.0 |
| concentrate | | |
| Fish meal¹ | 24.0 | 24.0 |
| Wheat gluten | 18.25 | 18.25 |
| Potato starch | 8.5 | 8.5 |
| Fish oil | 7.0 | 7.0 |
| rapeseed oil | 7.0 | 7.0 |
| Premix² | 0.8 | 0.8 |
| Hydrolysate | 0.0 | 1 |
| Lysine | 1.2 | 1.2 |
| Methionine | 0.7 | 0.7 |
| Monocalcium | 1.2 | 1.2 |
| phosphate | | |
| Yttrium oxide | 0.05 | 0.05 |
| Choline chloride | 0.3 | 0.3 |
| Gelatin | 5.0 | 5.0 |
| Total | 100 | 100 |

*% of dry matter relative to total weight of composition
¹Fish meal: from Pelagia Norway, reference: PA/ES/21/003, comprising 70.5% crude proteins and 9.4% crude fat.
²Premix: consists of a premix of vitamins and minerals comprising the following ingredients:
Vitamin A 500, 000 IU; Vitamin D3 300, 000 IU; Vitamin E 40,000 IU; Vitamin K3 2000 mg; Vitamin B1 3000 mg; Vitamin B2 5000 mg; Calcium D-pantothenate 8000 mg; Niacinamide 15, 000 mg; Vitamin B6 3000 mg; Folic acid 1000 mg; Vitamin B12 5000 mg; Vitamin C 25, 000 mg; Biotin 55,000 mg; calcium iodate, anhydrous, iodine 600.0 mg; manganese oxide, manganese 3,000.0 mg; zinc oxide, zinc 21000.0 mg.

TABLE 14
Gross composition of experimental diets

| | | Diet comprising |
|---|---|---|
| Gross | | hydrolysate |
| composition | | according to the |
| as % | CTRL2 | invention |
| Proteins | 51.3% | 51.3% |
| Lipids | 17.9% | 17.9% |
| Carbohydrates | 12.4% | 12.4% |
| Ash | 8.5% | 8.5% |

The diets detailed in Table 12 were manufactured by extrusion according to common techniques known to a person skilled in the art in order to obtain granules having a size of 2 mm.

The granules are obtained by mixing, firstly, soy protein concentrate, fish flour, wheat gluten, potato starch, premix, lysine, methionine, monocalcium phosphate, yttrium oxide, choline chloride according to the contents given in Table 13, in a Spiry 25 paste mixer. Secondly, the fish oil and the hydrolysate (for the diet according to the invention) were added to the mixture. Thirdly, activated gelatin at 60° C. was also added to the mixture. The resulting paste was converted into granules, cold, using an extruder. The granules were dried at 45° C. to obtain a dry matter content of about 93%.

Thus, in this embodiment, the hydrolysate is mixed with the other ingredients making up the feed (granule) before extrusion.

Each diet was tested in triplicate (3 tanks per diet). 180 Atlantic salmon with a mean weight of 20.3±0.3 g were distributed at random in each tank (6 tanks with a volume of 80 L each). The tanks are connected to a recirculating aquaculture system (RAS). The tanks are supplied with freshwater at a water temperature between 13.3 and 15.3° C. (with a mean temperature of 14.1° C.).

Each diet was given in excess of appetite and unconsumed feed was collected to accurately calculate the Feed Conversion Rate (FCR).

10 salmon per tank were selected in order to evaluate their growth performance. After 58 days of experimental feed, these 10 salmon were euthanized by anesthetic overdose.

b. Results

### Growth Evaluation

The 10 salmon selected were weighed at the start of the experiment and then at harvest (end of experiment). Their size was measured at harvest.

TABLE 15
Results on salmon growth performance

| Parameters | CTRL2 | Hydrolysate* |
|---|---|---|
| IBW (g) | 20.3 ± 0.3 | 20.3 ± 0.3 |
| FBW (g) | 87.6 ± 2.2 | 100.1 ± 2.8* |
| FTL (cm) | 18.4 ± 0.1 | 19.1 ± 0.1* |
| SGR (%/day) | 2.5 ± 0.02 | 2.8 ± 0.04* |
| FCR | 0.67 ± 0.005 | 0.67 ± 0.003 |

*By “Hydrolysate”, it is understood the diet comprising the hydrolysate according to the invention

The values are expressed as a mean±standard deviation (n=3). In this case, the standard deviation is equal to the standard error.

The asterisks indicate significant differences between the experimental groups and the CTRL2 control group (Student's t-test, P<0.05; n=3).

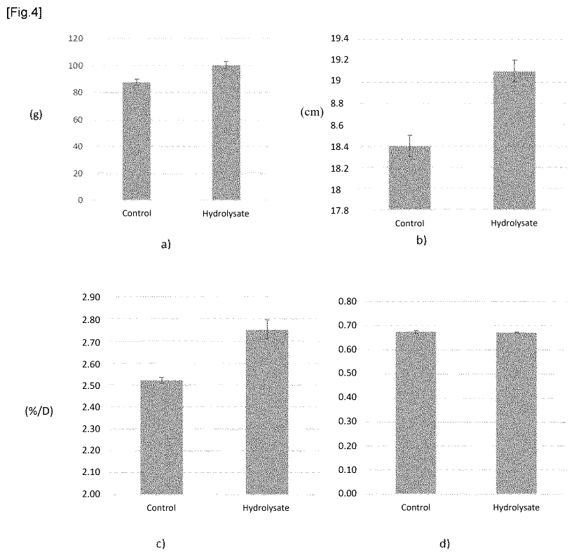

Based on the results presented above, it is observed that the body weight of the salmon fed with the granules comprising hydrolysate was multiplied 5-fold. Furthermore, it is observed that for an equivalent daily feed intake (FCR of 0.67 for the CTRL2 group and for the group fed with the diet comprising hydrolysate), the weight and length of salmon fed with the granules according to the invention increased significantly relative to salmon fed with CTRL2 granules. Indeed, a significant difference in body weight of 14.3% was obtained (see Table 15 and FIG. 4a).

### Fat Score Around Viscera and Eviscerated Weights

At the end of the experiment, the quantity of fat around the viscera of the salmon tested was determined by applying a visual determination method as detailed below:

The quantity of visceral fat was scored from 1 to 5 according to visibility of pyloric caeca (FIG. 5)).
a score of 1: pyloric caeca clearly visible,
a score of 2: pyloric caeca visible,
a score of 3: pyloric caeca visible in the form of cracks in visceral fat,
a score of 4: pyloric caeca visible through visceral fat,
a score of 5: pyloric caeca not visible.

Thus, the lower the score, the less the fat around the viscera.

Furthermore, the eviscerated weight of the salmon was also measured.

The results are shown in Table 16 below.

TABLE 16
Results on fat deposition around viscera

| Other parameters | CTRL2 | Hydrolysate* |
|---|---|---|
| Eviscerated weight | 77.9 ± 1.9 | 87.7 ± 2.2 |
| (g) | | |
| Fat around viscera | 1.8 ± 0.06 | 1.9 ± 0.07 |
| (score) | | |

*By “Hydrolysate”, it is understood the diet comprising the hydrolysate according to the invention

Based on these results, it is observed that although there is no significant difference between salmon fed with the granules of the CTRL2 group and those fed with granules according to the invention, the latter tend to have a higher eviscerated weight.

Furthermore, there is no significant difference in fat deposition around the viscera.

It can therefore be concluded from all these results that salmon fed with the granules according to the invention have significantly greater growth in terms of weight and length without becoming fatty. Thus, a diet for fish comprising the hydrolysate according to the invention specifically increases body muscle mass growth in addition to body length without causing fat accumulation around the viscera. Hence, the hydrolysate according to the invention makes it possible to considerably increase the growth of fish while keeping them in good physical condition, and thus improves the nutritional value of fish fed with granules comprising the hydrolysate according to the invention.

1. A hydrolysate obtained from insects, comprising at least 50% by weight of proteins, at least 7% by weight of lipids, at least 10.5% by weight of ash, the percentages by weight being given relative to the dry weight of the hydrolysate, and a dry matter content between 15 and 35% by weight, the percentage being given relative to the weight of the hydrolysate.

2. The hydrolysate according to claim 1, wherein the insects are Coleoptera.

3. The hydrolysate according to claim 1, comprising a fiber content of 1 to 5% by weight relative to the dry weight of the hydrolysate.

4. The hydrolysate according to claim 1, comprising water-soluble proteins, 75% of the water-soluble proteins having a size of less than 12,400 g/mol, relative to the total weight of water-soluble proteins.

5. The hydrolysate according to claim 1, comprising one or more additives chosen from a preservative and/or a pH-reducing agent.

6. The hydrolysate according to claim 1, comprising 40 to 60% by weight of insoluble proteins, the percentage by weight being expressed relative to the total weight of proteins.

7. A method for preparing a hydrolysate from insects comprising the following steps:
a) Separating the cuticles and the soft part of the insects,
b) Separating the soft part of the insects into an aqueous fraction, an oily fraction and a solid protein fraction, and
c) Enzymatic hydrolysis of the solid protein fraction.

8. The method according to claim 7, further comprising a step of slaughtering the insects, prior to the step of separating the cuticles from the soft part.

9. The method according to claim 7, further comprising a step of maturing the soft part of the insects, between the step of separating the cuticles from the soft part and the step of separating the soft part of the insects into an oily fraction, a solid fraction and an aqueous fraction.

10. The method claim 7, further comprising a step of stabilizing the hydrolyzed solid protein fraction obtained in step c), by adding one or more additives chosen from a preservative and/or a pH-reducing agent.

11. Method for feeding an animal comprising administering to an animal in need thereof the hydrolysate according to claim 1.

12. Method according to claim 11, wherein the feed is in the form of granules, flakes, paste, kibble, or treats.

13. Method according to claim 11, wherein the feed comprises from 1% to 20% by weight of the hydrolysate, the percentage by weight being given relative to the weight of the feed.

14. Method according to claim 11, wherein the animal is a farmed fish and wherein the method improves the growth of farmed fish.

15. Method according to claim 11, for improving fecal consistency.

16. An animal feed comprising 1 to 20% by weight of the hydrolysate according to claim 1, the percentage by weight being given relative to the weight of the feed.
